(19) United States
(12) Patent Application Publication
(10) Pub. No.: US 2010/0331645 A1
(43) Pub. Date: Dec. 30, 2010

(54) METHODS AND SYSTEMS FOR WIRELESS COMMUNICATION BETWEEN A BLOOD GLUCOSE METER AND A PORTABLE COMMUNICATION DEVICE

(75) Inventors: Joseph Michael Simpson, Fishers, IN (US); Michel Cadio, Carmel, IN (US); Blaine Edward Ramey, Indianapolis, IN (US); James D. Tenbarge, Fishers, IN (US); Michael J. Blackburn, Indianapolis, IN (US); Robert G. Davies, Carmel, IN (US); Carol J. Batman, Indianapolis, IN (US)

Correspondence Address: DINSMORE & SHOHL, LLP;FIFTH THIRD CENTER, ONE SOUTH MAIN STREET, SUITE 1300, DAYTON, OH, 45402, US

(73) Assignee: Roche Diagnostics Operations, Inc., Indianapolis, IN (US)

(21) Appl. No.: 12/491,306

(22) Filed: Jun. 25, 2009

(51) Int. Cl. A61B 5/1468

(52) U.S. Cl. 600/347

(57) ABSTRACT

A blood glucose measuring system which comprises a blood glucose (bG) meter and a portable communication device (PCD) and methods thereof are disclosed. The blood glucose meter comprises a measurement module and a wireless module, wherein the measurement module is operable to measure the blood glucose level of a blood sample, the wireless module is an embeddable module and communicates to the measurement module via a serial interface, and the wireless module is operable to wirelessly communicate to the portable communication device. The portable communication device is operable to wirelessly receive information from the blood glucose meter related to the blood glucose measurement.

### TECHNICAL FIELD

The present invention generally relates to methods and systems for wireless communication between a blood glucose meter and a portable communication device, and specifically, to methods and systems which wirelessly transmit the result of a blood glucose measurement from the meter to the portable communication device.

### BACKGROUND

As background, persons with diabetes suffer from either Type I or Type II diabetes in which the glucose level in the blood is not properly regulated by the body. As a consequence, many persons with diabetes often carry specialized electronic meters, called blood glucose (bG) meters, to allow them to periodically measure their glucose level and take appropriate action, such as administering insulin. In addition to the bG meter, users may also carry a portable communication device (PCD), such as a cellular phone, smart phone, personal digital assistant (PDA), or similar device.

Often people rely on their PCD as the primary means for planning, scheduling, and communicating. As a result, most PCDs are equipped with a variety of application software which provides a powerful and user-friendly means for viewing and/or inputting data. For example, many PCDs contain a “calendar” function which permits the user to input appointments as well as alerts the user when appointments are due.

Accordingly, a person with diabetes may wish to wirelessly transmit the result of a bG measurement from his bG meter to his PCD in order to, for example, display and/or store the bG measurement result.

### SUMMARY

In one embodiment, a blood glucose measuring system comprises a blood glucose (bG) meter and a portable communication device (PCD). The blood glucose meter comprises a measurement module and a wireless module, wherein the measurement module measures the blood glucose level of a blood sample, the wireless module is an embeddable module and communicates to the measurement module via a serial interface and wirelessly to the portable communication device. The portable communication device receives information from the blood glucose meter related to the blood glucose measurement.

In another embodiment, a method is disclosed for establishing wireless communication between a blood glucose (bG) meter and a portable communication device (PCD), wherein the bG meter comprises a measurement module and a wireless module, wherein the measurement module measures the blood glucose level of a blood sample, and the wireless module is an embeddable module and communicates to the measurement module via a serial interface. The method comprises switching on power to the bG meter and the PCD, sending a pairing beacon by the bG meter to the PCD, wherein the pairing beacon comprises information related to a type of the bG meter and a serial number of the bG meter, receiving the pairing beacon by the PCD, determining whether the PCD will establish wireless communication to the bG meter based on the type and the serial number of the bG meter, sending a response to the pairing beacon by the PCD to the bG meter indicating whether the PCD will establish wireless communication to the bG meter, and indicating on the bG meter whether the wireless communication was established.

In yet another embodiment, a method is disclosed for wirelessly communicating a result of a blood glucose measurement between a blood glucose (bG) meter and a portable communication device (PCD), wherein the bG meter comprises a measurement module and a wireless module, wherein the measurement module measures the blood glucose level of a blood sample, and the wireless module is an embeddable module and communicates to the measurement module via a serial interface. The method comprises inserting a measurement strip into the bG meter, wherein the measurement strip has a sample of the blood to be measured, measuring the bG level of the blood sample by the measurement module of the bG meter, serially sending a result of the bG level measurement by the measurement module to the wireless module, wirelessly sending the result of the measurement by wireless module of the bG meter to the PCD, and displaying the result of the measurement on the PCD.

These and additional features provided by the embodiments of the present invention will be more fully understood in view of the following detailed description, in conjunction with the drawings.

### BRIEF DESCRIPTION OF THE DRAWINGS

The embodiments set forth in the drawings are illustrative and exemplary in nature and not intended to limit the inventions defined by the claims. The following detailed description of the illustrative embodiments can be understood when read in conjunction with the following drawings, where like structure is indicated with like reference numerals and in which:

FIG. 1 depicts a blood glucose measuring system according to one or more embodiments shown and described herein;

FIG. 2 depicts a measurement module of a bG meter according to one or more embodiments shown and described herein;

FIG. 3 depicts a wireless module of a bG meter according to one or more embodiments shown and described herein;

FIG. 4 depicts a method of establishing communication between a bG module and a PCD according to one or more embodiments shown and described herein;

FIG. 5 depicts a method of transmitting the result of a blood glucose measurement from a bG meter to a PCD according to one or more embodiments shown and described herein;

FIG. 6 depicts a method of setting up operating parameters of the bG meter and/or PCD according to one or more embodiments shown and described herein;

FIG. 7 depicts the transmission of messages between the measurement module and the communication module according to one or more embodiments shown and described herein; and

FIG. 8 depicts the transmission of messages between the measurement module and the communication module according to one or more embodiments shown and described herein.

### DETAILED DESCRIPTION

The embodiments described herein generally relate to methods and systems for wireless communication between a blood glucose meter and a portable communication device, and specifically, to methods and systems which wirelessly transmit the result of a blood glucose measurement from the bG meter to the portable communication device.

For the purposes of this specification, wireless communication refers to the transmission of data or information without the use of physical conductors or “wires.” One type of wireless communication may be radio frequency, or “RF,” in which the data is transmitted via electromagnetic waves. For example, “Bluetooth” is one type of wireless RF communication system which uses a frequency of approximately 2.4 Gigahertz (GHz). Another type of wireless communication scheme may use infrared light, such the systems supported by the Infrared Data Association (IrDA). Other types of wireless communication are also contemplated, including present technologies and yet-to-be developed technologies.

Also for the purposes of this specification, a portable communication device (PCD) may include any type of battery-powered device which permit people to wirelessly communicate via voice and/or data to other people. Examples of PCDs include but are not limited to cellular phones, smart phones, and personal digital assistants (PDAs). Many of these PCDs may use a wireless cellular network, such as the 3G (third generation) telecommunications standard. Other wireless networks and/or communication schemes may be used, as is known in the art.

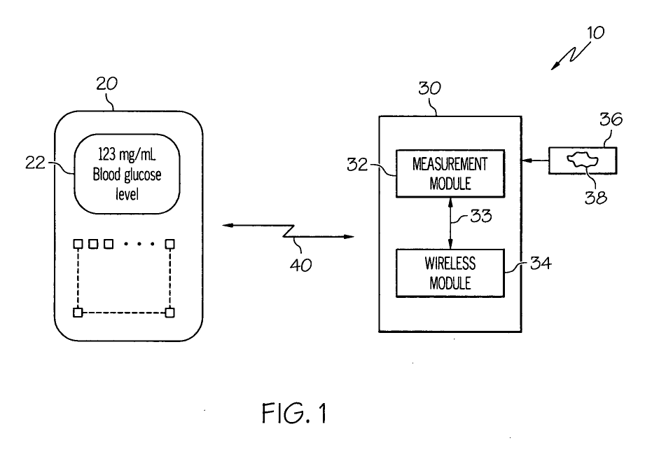

FIG. 1 shows one embodiment of a blood glucose measuring system 10 comprising a portable communication device (PCD) 20 and a blood glucose (bG) meter 30. The PCD 20 and bG meter 30 may linked via a wireless communication system 40. The PCD 20 may comprise a display 22, which is capable of displaying information to the user. The display may be a liquid crystal display (LCD) or other type of display, as is known in the art. The bG meter 30 may comprise a measurement module 32 and a wireless module 34. The measurement module is capable of measuring the blood glucose level of a blood sample 38 placed on an insertable measurement strip 36. When the user wishes to measure his blood glucose level, he inserts the measurement strip 36 into the bG meter 30 and places a blood sample 38 on the measurement strip 36. The wireless module 34 may be embedded in the bG meter 30 and may be electrically connected to the measurement module 32 via a serial interface 33.

Still referring to FIG. 1, in one embodiment the PCD 20 contains the appropriate hardware and software which permit it to wirelessly communicate to the bG meter 30 via the wireless communication system 40. In the bG meter 30, the wireless module 34 in one embodiment likewise contains the appropriate hardware and software which permit the bG meter 30 to wirelessly communicate to the PCD 20 via the wireless communication system 40. Such hardware (for either the PCD or the bG meter) may include an oscillator, an antenna, light-emitting diodes, capacitors, resistors, inductors, or any other component which may be suitable for facilitating the wireless communication system 40. In addition, the software (for either the PCD or the bG meter) may include computer programs, device drivers, or any other suitable software for facilitating the wireless communication system 40. For example, the software may comprise a data link layer, a network layer, a transport layer, and/or an application layer.

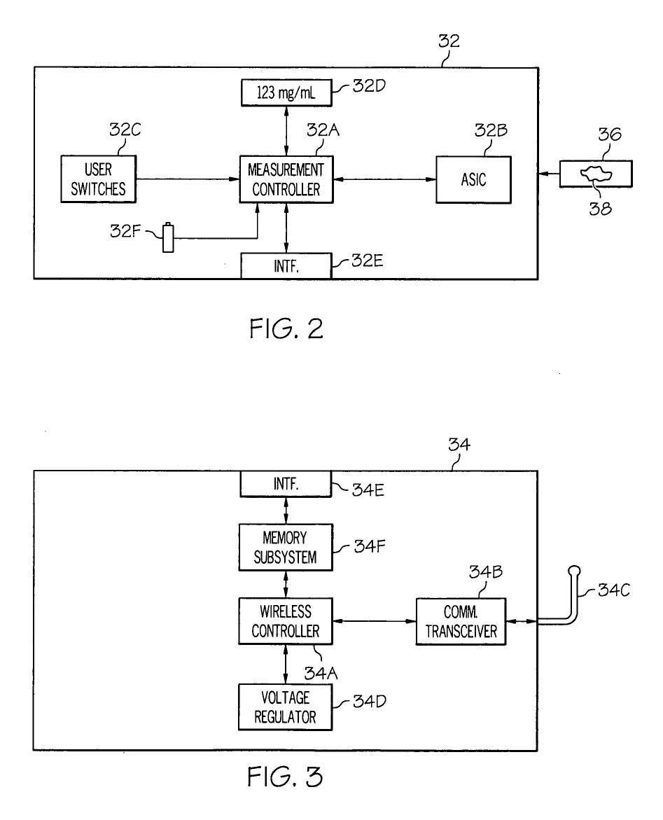

FIG. 2 depicts a block diagram of the hardware architecture of the measurement module 32 according to one embodiment. The measurement module in one embodiment comprises a measurement controller 32A, a measuring ASIC (application-specific integrated circuit) 32B, user switches 32C, a display 32D, an interface 32E to the communication module, and a battery 32F. The measurement controller 32A may comprise a microcontroller, a microprocessor, a digital signal processor, or other suitable device. The measurement controller 32A in one embodiment is in electrical communication with the measuring ASIC 32B which may be operable to perform blood glucose measurements of a blood sample 38 placed on a measurement strip 36. The measurement controller 32A in another embodiment may also be in electrical communication with the user switches 32C which permits the user to set certain operating conditions for the bG meter. For example, one of the switches may allow the user to set the type of PCD to which the bG meter may communicate. Other switches may control the operation of the software.

Also as shown in FIG. 2, the measurement controller 32A in one embodiment is in electrical communication with a display 32D. The display 32D may be a liquid crystal display (LCD), eight-segment LED digits, or other suitable displays. It may be operable to display information about the operation of the bG meter, such as the result of a previous bG measurement, the time of day, or the state of the battery 32F. The display 32D may automatically turn off after some period of non-use in order to conserve battery power. The measurement controller 32A in another embodiment may also be in electrical communication with an interface 32E to the wireless module. This interface 32E may be a serial interface and may comprise a connector which may matingly engage a connector on the wireless module. The interface 32E may employ a variety of wired protocols, including but not limited to a USART (universal synchronous/asynchronous receiver and transmitter), an SPI (Serial Peripheral Interface), or the I²C (Inter-Integrated Circuit) interface. In addition, the interface 32E may employ a wireless protocol, such as infrared. Other types of serial protocols may be used as is known in the art. The battery 32F may provide power to the other elements in the measurement module 32, such as the measurement controller 32A, the measuring ASIC 32B, and so forth. Other electrical components not specifically mentioned here may also be used in the measurement module 32, such as but not limited to oscillators, resistors, capacitors, inductors, etc.

FIG. 3 illustrates a block diagram of the hardware architecture of the wireless module 34 according to one embodiment. The wireless module 34 may comprise a communication controller 34A, a communication transceiver 34B, an antenna 34C, a voltage regulator 34D, an interface 34E to the measurement module, and a memory subsystem 34F. The communication controller 34A may comprise a microcontroller, a microprocessor, a digital signal processor, or other suitable device, and may be electrical communication with the communication transceiver 34B such that the data to be transmitted from the bG meter to the PCD may be sent from the communication controller 34A to the communication transceiver 34B. Conversely, data transmitted from the PCD to the bG meter may be received by the communication transceiver 34B and sent to the communication controller 34A. The communication transceiver 34B may comprise a microcontroller, such as one from the 8051 family of microcontrollers, and an RF transceiver integrated circuit, such as the nRF24L01+ from Nordic Semiconductor, Inc. The antenna 34C may be used if an RF system is used as the wireless communication scheme between the bG meter and the PCD. If an infrared communication scheme is used instead, the antenna 34C may be replaced by an infrared light generator/detector circuit. The antenna 34C may be contained within the housing of the bG meter so it is hidden from the user. Alternatively, the antenna 34C may be partially or fully external to the housing of the bG meter and, thus, may be visible by the user.

Still referring to FIG. 3, the voltage regulator 34D in one embodiment receives a voltage from the measurement board and regulates the voltage to a level suitable for the wireless module 34. For example, the measurement module may provide 3.0 Volts to the wireless module in order to provide power for its circuitry. The voltage regulator 34D in one embodiment regulates this voltage to 1.8 Volts such that the communication controller 34A, the communication transceiver 34B, and other circuitry on the wireless module 34 operate at about 1.8 Volts. The voltage regulator 34D may thus provide a relatively constant voltage to the electronic circuitry since the battery voltage may drop over time as the battery wears out. In one embodiment, the memory subsystem 34F may be omitted, and the communication controller 34A may be in direct electrical communication with an interface 34E to the measurement module. This interface 34E may be a serial interface and may comprise a connector which may matingly engage a connector on the measurement module. As discussed above, the interface 34E may employ a variety of wired protocols, such as a USART, an SPI interface, or an I²C interface. In addition, the interface 34E may employ a wireless protocol, such as infrared. Other types of serial protocols may be used as is known in the art.

In an alternative embodiment, the wireless module 34 may further comprise a memory subsystem 34F that temporarily stores data moving between the measurement module and the wireless module 34. In some embodiments, the memory subsystem 34F does not control other circuitry, and in some such embodiments the memory subsystem 34F may be provided in the form of a conventional memory device (e.g., a static random access memory (RAM)). In other embodiments in which the memory subsystem 34F does or does not control other circuitry, the memory subsystem 34F may be provided in the form of a conventional processor that is configured to operate as a Dual-Port RAM (DPR) processor. In such embodiments, the DPR processor operates from a clock signal that is separate from clock signal from which the communication controller 34A operates. In one embodiment, such a DPR processor is a MC9S08GT16A 8-bit microcontroller unit that is commercially available from Freescale Semiconductor, Inc. of Austin, Tex., although this disclosure contemplates other implementations of the memory subsystem 34F that is provided in the form of a conventional processor configured as a DPR processor.

Referring again to FIG. 1, the bG meter in the illustrated embodiment comprises a measurement module 32 and a wireless module 34. Both the measurement module 32 and the wireless module 34 may be installed in the housing of the bG meter 30. The wireless module 34 may be embedded in the bG meter 30 such that it may by physically and electrically coupled to the measurement module 32 through an electrical connector. For example, this connector may comprise one mating portion on the measurement module 32 and the other mating portion on the wireless module 34. In addition, this exemplary connector may contain a suitable number of conductors (e.g., connector pins and/or sockets) in order to permit the serial communication between the measurement module 32 and the wireless module 34. Furthermore, the exemplary connector may contain a suitable number of conductors to permit the measurement module 32 to supply power to the wireless module 34 by way of, as an example, voltage from a battery. Other types of connectors and methods of connecting the two modules may be used, as is known in the art.

Although not shown in FIG. 1, the bG meter 30 may also comprise other features and/or functions, such as but not limited to pushbuttons, a display, light emitting diodes (LEDs), and a measurement strip interface. The bG meter may include pushbuttons to allow the user to turn the meter on and off, input data, set operating parameters, and so forth. The display may permit the user to view information about the meter or about the result of a blood glucose measurement. Furthermore, the display may indicate whether the meter has successfully established a connection to the PCD. The LEDs on the bG meter 30 may indicate whether the meter is turned on and/or whether the battery is low. The bG meter 30 may also contain a measurement strip interface which allows the user to insert a measurement strip 36 containing a blood sample 38. This measurement strip interface may comprise a slot in the side of the meter or any other suitable means for “reading” the blood sample 38 contained on the measurement strip 36. As indicated above, the bG meter may contain these and other features which facilitate the operation of the meter for the user. Other embodiments of the bG meter may omit some or all of these features. For example, since information about the bG meter and the result of the blood glucose measurement may be transmitted to and displayed by the PCD, the bG meter may omit the display.

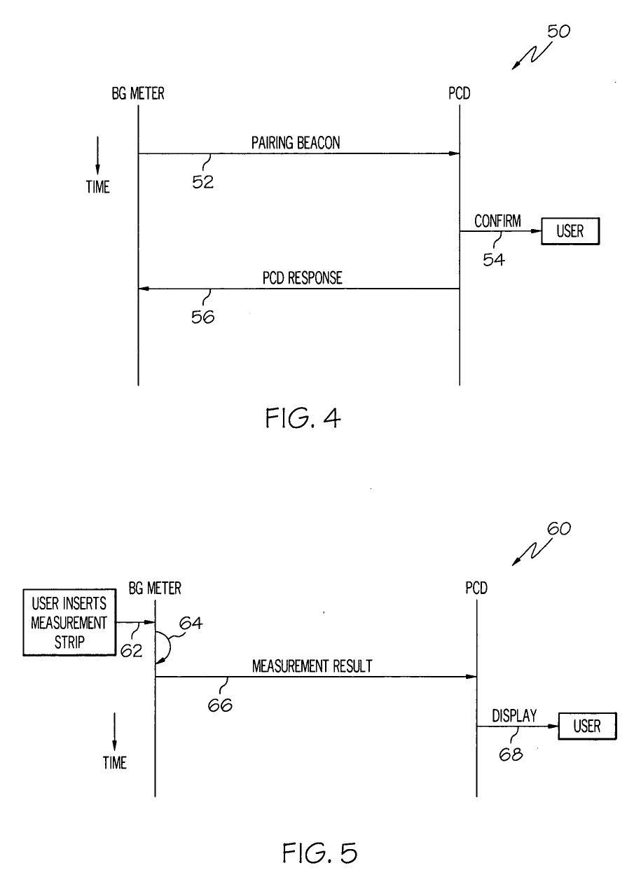

FIG. 4 depicts one embodiment of establishing a communication link between the bG meter and the PCD. Because many people carry PCDs and some of these people may also carry a bG meter, it is possible that two bG meters and two PCDs may come in close proximity to each other. Consequently, it may be useful to establish a unique wireless communication link between a user's PCD and bG meter so that bG meters carried by other people may not interfere with the operation of the user's bG meter. After switching on the power to the bG meter and the PCD, the bG meter may transmit a pairing beacon 52 to the PCD. The pairing beacon 52 may contain information regarding the type of bG meter (e.g., model number) as well as the serial number of the bG meter. The pairing beacon 52 may contain other information related to the bG meter as well such as, for example, the manufacturer, the status of the battery, and/or the result of the previous blood glucose measurement. Upon receiving the pairing beacon 52, the PCD may determine, based on the information contained in the pairing beacon 52, whether to establish communication with the bG meter. The PCD may then transmit a response 56 to the bG meter which indicates whether communication was established or not.

In another embodiment, the PCD may pose a confirmation request 54 to the user, which would allow the user the option of proceeding with or cancelling the establishment of communication. As an example, the confirmation request 54 may appear on the display of the PCD and may indicate the model number and serial number of the user's bG meter. The user may observe this displayed information and determine whether to permit the establishment of communication.

In still another embodiment, the PCD may already contain the model number and serial number of the bG meter. This information may be input by the user to the PCD during a setup procedure. In this embodiment, communication between the PCD and the bG meter may automatically be established, without any input from the user, since the PCD already has the information which determines whether the link will be established or not.

The establishment of communication (between the bG module and the PCD) may conform with the requirements of the communication technology being used. For example, if the Bluetooth system is used for the wireless communication, the bG meter and the PCD may be required to conform to the requirements of the Bluetooth specification. As an example, the Bluetooth specification may require that the pairing beacon be transmitted at a specific frequency, while subsequent messages be transmitted at a different frequency. Furthermore, the bG module and/or the PCD may include a timer which requires the communication to be established within a predetermined time interval, such as 60 seconds. If the link is not established before the expiration of this timer, further communication may be prohibited until the power on the PCD and/or bG meter is cycled.

The transmitted pairing beacon 52 may comprise a number of bytes which relate to information about the bG meter. For example, the pairing beacon 52 may comprise six bytes for the serial number of the meter and two bytes for the module number of the meter. Additionally, two bytes may be used to indicate the version number of the software and/or hardware. Still other bytes may represent the version of the wireless interface between the bG meter and the PLD.

Upon establishment of communication, the bG module and the PCD may be able to transmit data to each other. This communication link may still exist, even though it may be temporarily unable for data to be transmitted due to, for example, the devices being out of range or a third device interfering with the communication link. In these cases, the devices may automatically re-establish the link once they are back in range or the interfering device is removed.

FIG. 5 illustrates the steps for a user-initiated blood glucose measurement 60 according to one embodiment of the bG meter and PCD system. At step 62, the user may insert a measurement strip into the bG meter wherein the measurement strip contains the blood sample to be measured. The bG meter may take the measurement at step 64, which may require some processing time by the measurement module. At step 66, the result of the blood glucose measurement may be wirelessly transmitted to the PCD. If the bG meter and the PCD are not able to immediately communicate with each other due to, for example, being out of range, the bG meter may store the result and periodically attempt to transmit the result to the PCD. At step 68, the result of the blood glucose measurement may be displayed by the PCD. As an alternative embodiment, the user may press a button on the bG meter indicating that he wishes to take a measurement, at which time the bG meter may prompt the user to insert the measurement strip. In still another embodiment, the bG meter or PCD may periodically prompt or remind the user to take a blood glucose measurement based on, for example, the amount of time from the previous measurement.

The transmitted result of the blood glucose measurement (transmitted at step 66) may comprise a number of bytes related to the result. For example, two bytes may represent the result itself (e.g., 0 to 999 mg/dL), two bytes may identify the measurement strip, five bytes may represent the result's timestamp (e.g., date and time), and one byte may be used for flags (e.g., low battery). Other bytes may be used as is known in the art.

Before the result of the blood glucose measurement is transmitted to the PCD, the bG meter may store the result (and its corresponding timestamp) in its non-volatile memory. Furthermore, the bG meter may store any number of previous results. For example, the bG meter may store the previous 500 results in its non-volatile memory. Likewise, after the result is transmitted to the PCD, the PCD may store the result in its non-volatile memory. In addition, the PCD may store any number of previous results. Furthermore, the PCD may wireless transmit a result to another device, person, or database via its wireless network (e.g., 3G cellular network). For example, the PCD may transmit the result to a database accessible by the user and/or the user's health care provider. In this fashion, a historical record of the results of the blood glucose measurements can be automatically maintained. As another example, if the result exceeds a predetermined value, the PCD may send the result and/or a message directly to the user's health care provider. In addition to the result of the blood glucose measurement, the PCD may also contemporaneously transmit (to another device, person, or database) additional information, such as but not limited to the timestamp of the result or the location of the user (e.g., from a global positioning system or GPS).

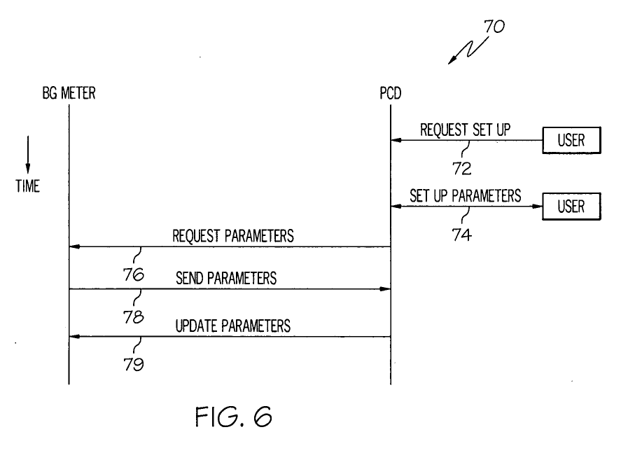

FIG. 6 illustrates the steps for a user-initiated setup procedure 70 according to one embodiment of the bG meter and PCD system. At step 72, the user may request that the system be set up via the PCD. Such a set up procedure may permit the user to adjust some or all of the operating parameters of the system, including but not limited to whether and how often the user is reminded to take a blood glucose measurement or whether each blood glucose result is wirelessly transmitted (via the PCD) to a database. Other operating parameters may be set up or adjusted as well, as is known in the art. At step 74, the display of the PCD may indicate (textually or graphically) which parameter is being adjusted. Also at this step, the user may indicate (by using the inputting means of the PCD) which parameters, if any, he wishes to adjust. Some of the parameters may ultimately be stored in the PCD while others may be stored on the bG meter. At step 76, the PCD may request the value of the parameter if it is stored on the bG meter. In response, the bG meter may transmit the value of the parameter to the PCD at step 78. Alternatively, the value of the parameter, if stored on the PCD, may simply be retrieved by the PCD from its internal memory. Finally, at step 79, adjusted parameters which are stored on the bG meter may be transmitted to the bG meter for non-volatile storage. Alternatively, adjusted parameters which are to be stored on the PCD may simply be permanently stored by the PCD in its internal memory.

One parameter which may be adjusted via the setup procedure 70 is the synchronization of the time-of-day clock between the bG meter and the PCD. The time-of-day clock maintains the current the year, month, day, hour, minute, and second. This information may help “timestamp” each blood glucose measurement. In one embodiment, both the bG meter and the PCD may contain a time-of-day clock. Due to variations in electrical components, these two clocks may not match precisely. The user may have the ability to determine when and/or how often the two clocks are synchronized. For example, the user may specify that the bG time-of-day clock be adjusted to match the PCD time-of-day clock every time a blood glucose measurement is performed. Alternatively, the bG clock may be adjusted to match the PCD clock at specific intervals, such as every hour. It should be noted that the PCD clock may also be periodically synchronized to a time-of-day clock from another source, such as the cellular phone service provider.

The communication link between the bG meter and PCD may be disestablished by a number of methods. First, either the bG meter and/or the PCD may be switched off. Second, the user may request either to the bG meter or PCD that the link be disestablished. On the bG meter, this may be accomplished, for example, by pressing a button. On the PCD, this may be accomplished, for example, by pressing a button or by selecting an appropriate set up procedure.

Although at least two basic categories of messages (result of blood glucose measurement and setup procedures) have been described herein, it is contemplated that other types of messages may be used as well. These may include messages which facilitate the fundamental operation of the wireless communication link, called physical-layer (PL) messages. For example, a PL message may be sent by either the PCD or the bG meter which asks the other device to increase its power level for transmitting messages. This message, in turn, may be transmitted due to poor quality of a received message. Furthermore, application-layer (AL) messages may be sent as well. These may include, as an example, an AL message from the PCD to the bG meter asking for the status of its battery. Other AL messages may include a request to transmit historical information from the PCD to the bG meter or a request to temporarily suspend communication (due to, for example, regulations when traveling on a plane). Both PL and AL messages may comprise a suitable number of data bytes, as is known in the art. In short, many types of messages not explicitly described herein may be transmitted between the PCD and the bG meter.

Furthermore, the communication link may employ security means in order to discourage other people from viewing and/or modifying the messages. As an example, the PCD and bG meter may use technology in order to encrypt the messages set between them. Such a system may use encryption technology that is known in the art or is yet to be developed. This type of security may be sufficient to satisfy any state or federal regulations which require that health care information be kept secure.

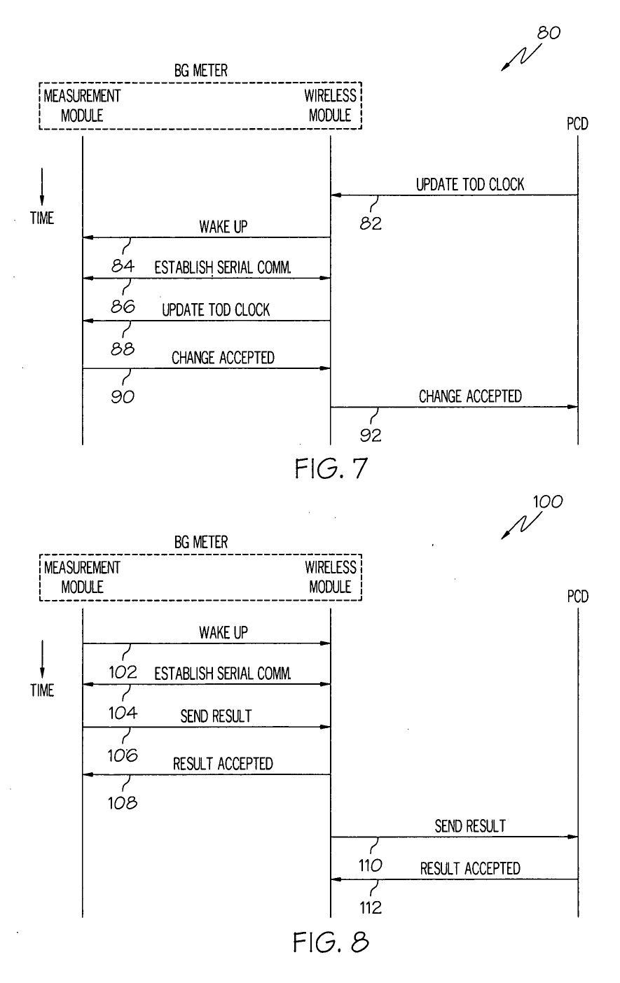

In addition to the wireless communication between the bG meter and the PCD, there may be serial communication between the measurement module and the wireless module of the bG meter. Since the wireless module is an embeddable module, and since both the measurement module and the wireless module each have their own respective controllers, they may operate independently of each other. FIG. 7 depicts one embodiment of the communication 80 between the measurement module and the wireless modules after the bG meter receives a command from the PCD to update the time-of-day (TOD) clock. At step 82, the PCD sends a command to the bG meter to update its TOD clock. The wireless module receives the message and, at step 84, sends a message to the measurement module to wake up (from sleep mode). At step 86, the wireless module and the measurement module may establish a serial communication link. At step 88, the wireless module may send a message to the measurement module to change the TOD clock (which may reside on the measurement module). At step 90, the measurement module may send a message to the wireless module indicating that it accepted the change. Finally, at step 92, the wireless module may send a message to the PCD indicating that the bG meter has accepted the TOD update. Upon completion of these tasks, the measurement and/or wireless module may go into “sleep mode” in which its power consumption is reduced.

FIG. 8 illustrates one embodiment of the communication 100 between the measurement module and the wireless modules after the user inserts a measurement strip (containing the blood sample) into the bG meter. At step 102, the measurement module requests that the wireless module wake up (from sleep mode). At step 104, the wireless module and the measurement module may establish a serial communication link. At step 106, the measurement module may send the result of the blood glucose measurement to the wireless module. The wireless module may indicate that it has accepted the result at step 108. The result of the blood glucose measurement may be sent by the bG meter (via the wireless module) to the PCD at step 110. Finally, at step 112, the PCD may indicate to the bG meter that it has accepted the result. Upon completion of these tasks, the measurement and/or wireless module may go into “sleep mode” in which its power consumption is reduced. The communication schemes described in FIGS. 7 and 8 are merely intended to show examples of the types of messages that may be transmitted between the measurement module and wireless module. Other messages may be transmitted, as is known in the art.

Although the embodiments described herein indicate that a single bG meter may wirelessly communicate with a PCD, it is also contemplated that additional meters and/or electronic devices may also wirelessly communicate with the same PCD at the same time. For example, a user may have a PCD, a bG meter, and the blood pressure meter. Both the bG meter and/or the blood pressure meter may wireless communicate with the PCD. As another example, a user may have a PCD and two bG meters (e.g., one for himself and one for his spouse or child). In this case, both bG meters may wirelessly communicate with the PCD at the same time.

It should now be understood that the methods and systems described herein may permit wireless communication between a blood glucose (bG) meter and a portable communication device (PCD). Specifically, the methods may allow the bG meter and the PCD to initially establish a communication link. Furthermore, the methods may also allow the bG meter to transmit the result of a blood glucose measurement to the PCD, which may display the result of the measurement to the user.

While particular embodiments and aspects of the present invention have been illustrated and described herein, various other changes and modifications may be made without departing from the spirit and scope of the invention. Moreover, although various inventive aspects have been described herein, such aspects need not be utilized in combination. It is therefore intended that the appended claims cover all such changes and modifications that are within the scope of this invention.

What is claimed is:

1. A blood glucose measuring system comprising a blood glucose (bG) meter and a portable communication device (PCD), wherein:
the blood glucose meter comprises a measurement module and a wireless module, wherein:
the measurement module measures the blood glucose level of a blood sample;
the wireless module is an embeddable module and communicates to the measurement module via a serial interface and wirelessly to the portable communication device; and
the portable communication device receives information from the blood glucose meter related to the blood glucose measurement.

2. The system of claim 1 wherein the wireless communication is Bluetooth, Zigbee, or infrared light.

3. The system of claim 1 wherein the PCD is a cellular phone, a smart phone, or a personal digital assistant.

4. The system of claim 1 wherein the measurement module comprises a measurement controller, user switches, a measurement application-specific integrated circuit (ASIC), a display, a serial interface to the communication module, and a battery.

5. The system of claim 4 wherein the measurement ASIC receives a measurement strip and determines the blood glucose level of a blood sample placed on the measurement strip.

6. The system of claim 1 wherein the communication module comprises a communication controller, a communication transceiver, an antenna, a voltage regulator, and an interface to the measurement module.

7. A method for establishing wireless communication between a blood glucose (bG) meter and a portable communication device (PCD), wherein the bG meter comprises a measurement module and a wireless module, wherein the measurement module measures the blood glucose level of a blood sample, and the wireless module is an embeddable module and communicates to the measurement module via a serial interface, the method comprising:
switching on power to the bG meter and the PCD;
sending a pairing beacon by the bG meter to the PCD, wherein the pairing beacon comprises information related to a type of the bG meter and a serial number of the bG meter;
receiving the pairing beacon by the PCD;
determining whether the PCD will establish wireless communication to the bG meter based on the type and the serial number of the bG meter;
sending a response to the pairing beacon by the PCD to the bG meter indicating whether the PCD will establish wireless communication to the bG meter; and
indicating on the bG meter whether the wireless communication was established.

8. The method of claim 7 wherein the sending of the pairing beacon and the sending of the response to the pairing beacon are performed by way of Bluetooth, Zigbee, or infrared light.

9. The method of claim 7 wherein the PCD is one of a cellular phone, a smart phone, and a personal digital assistant.

10. The method of claim 7 wherein the measurement module comprises a measurement controller, user switches, a measurement application-specific integrated circuit (ASIC), a display, a serial interface to the communication module, and a battery.

11. The method of claim 10 further comprising determining the blood glucose level of a blood sample by providing a measurement strip to the measurement ASIC and providing the blood sample to the measurement strip.

12. The method of claim 7 wherein the communication module comprises a communication controller, a communication transceiver, an antenna, a voltage regulator, and an interface to the measurement module.

13. A method for wirelessly communicating a result of a blood glucose measurement between a blood glucose (bG) meter and a portable communication device (PCD), wherein the bG meter comprises a measurement module and a wireless module, wherein the measurement module measures the blood glucose level of a blood sample, and the wireless module is an embeddable module and communicates to the measurement module via a serial interface, the method comprising:
inserting a measurement strip into the bG meter, wherein the measurement strip has a sample of the blood to be measured;
measuring the bG level of the blood sample by the measurement module of the bG meter;
serially sending a result of the bG level measurement by the measurement module to the wireless module;
wirelessly sending the result of the measurement by wireless module of the bG meter to the PCD; and
displaying the result of the measurement on the PCD.

14. The method of claim 13 wherein the sending of the pairing beacon and the sending of the response to the pairing beacon are performed by way of Bluetooth, Zigbee, or infrared light.

15. The method of claim 13 wherein the PCD is selected from a cellular phone, a smart phone, and a personal digital assistant.

16. The method of claim 13 wherein the measurement module comprises a measurement controller, user switches, a measurement application-specific integrated circuit (ASIC), a display, a serial interface to the communication module, and a battery.

17. The method of claim 16 wherein the measurement ASIC determines the blood glucose level of the blood sample from the measurement strip inserted into the bG meter.

18. The method of claim 13 wherein the communication module comprises a communication controller, a communication transceiver, an antenna, a voltage regulator, and an interface to the measurement module.
